(19) United States
(12) Patent Application Publication
(10) Pub. No.: US 2015/0351639 A1
(43) Pub. Date: Dec. 10, 2015

(54) SUBJECT INFORMATION OBTAINING APPARATUS, METHOD FOR CONTROLLING SUBJECT INFORMATION OBTAINING APPARATUS, AND PROGRAM

(71) Applicant: CANON KABUSHIKI KAISHA, Tokyo (JP)

(72) Inventor: Hiroshi Abe, Kyoto-shi (JP)

(21) Appl. No.: 14/655,965

(22) PCT Filed: Dec. 3, 2013
(86) PCT No.: PCT/JP2013/007093

§ 371 Date: Jun. 26, 2015

(30) Foreign Application Priority Data
Dec. 28, 2012 (JP) 2012-286686

(51) Int. Cl. A61B 5/00

(52) U.S. Cl. CPC A61B 5/0095; A61B 5/7246

(57) ABSTRACT

A subject information obtaining apparatus includes a light source that emits light, a photoacoustic wave reception unit that receives a photoacoustic wave generated when the light is radiated onto a subject and that outputs a photoacoustic signal, an acoustic wave transmission unit that transmits an acoustic wave to the subject, an echo reception unit that receives an echo of the acoustic wave and that outputs an echo signal, and a signal processing unit that obtains pieces of optical characteristic information regarding the subject on the basis of the photoacoustic signal and pieces of morphological information regarding the subject on the basis of the echo signal. The signal processing unit obtains similarity between the pieces of morphological information, and, if the similarity is equal to or higher than a certain value, combines the pieces of optical characteristic information corresponding to the pieces of morphological information.

### TECHNICAL FIELD

The present invention relates to a subject information obtaining apparatus that obtains information regarding a subject using acoustic waves, a method for controlling the subject information obtaining apparatus, and a program.

### BACKGROUND ART

Optical imaging apparatuses that radiate light onto living bodies from light sources such as lasers and that image information regarding the insides of the living bodies obtained on the basis of the incident light are being developed in the medical field. As one of optical imaging technologies, there is photoacoustic imaging (PAI). In the PAI, pulse light generated by a light source is radiated onto a living body, and photoacoustic waves generated by tissue, which has absorbed the energy of the pulse light that has propagated through and diffused by the living body, are received. Optical characteristic information regarding the inside of the living body is then imaged on the basis of reception signals of the photoacoustic waves.

Here, the optical characteristic information includes, for example, initial sound pressure distribution, light absorption energy density distribution, and light absorption coefficient distribution. These pieces of information may be used for measuring the concentration of a substance (for example, hemoglobin concentration in blood, oxygen saturation of blood, or the like) in a subject when the measurement is conducted using light having various wavelengths.

However, reception signals of photoacoustic waves include noise caused by various factors. As a result, the signal-to-noise (SN) ratio of the reception signals decreases, thereby decreasing the quantitativity of optical characteristic information imaged using the reception signals.

Therefore, in NPL 1, a method for improving the quantitativity of optical characteristic information by obtaining an arithmetic mean of a plurality of pieces of photoacoustic characteristic information is disclosed.

### CITATION LIST

### Non Patent Literature

NPL 1: M. Jaeger et al “Improved Contrast Deep Optoacoustic Imaging Using Displacement-Compensated Averaging: Breast Tumour Phantom Studies”, Phys. Med. Biol. 56, 5889 (2011)
NPL 2: Y. Yamada et al. “Light-Tissue Interaction and Optical Imaging in Biomedicine”, Journal of Mechanical Engineering Laboratory, January 1995, Vol. 49, No. 1, pp. 1-31

### SUMMARY OF INVENTION

### Solution to Problem

A subject information obtaining apparatus disclosed herein includes a light source configured to emit light, a photoacoustic wave reception unit configured to receive a photoacoustic wave generated when the light is radiated onto a subject and output a photoacoustic signal, an acoustic wave transmission unit configured to transmit an acoustic wave to the subject, an echo reception unit configured to receive an echo of the acoustic wave and output an echo signal, and a signal processing unit configured to obtain a plurality of pieces of optical characteristic information regarding the subject on the basis of the photoacoustic signal and a plurality of pieces of morphological information regarding the subject on the basis of the echo signal. The signal processing unit obtains similarity between the plurality of pieces of morphological information. If the similarity is equal to or higher than a certain value, the signal processing unit combines the plurality of pieces of optical characteristic information corresponding to the plurality of pieces of morphological information.

Further features of the present invention will become apparent from the following description of exemplary embodiments with reference to the attached drawings.

### BRIEF DESCRIPTION OF DRAWINGS

FIG. 1 is a diagram illustrating a subject information obtaining apparatus according to an embodiment of the present invention.

FIG. 2 is a diagram illustrating details of a signal processing unit according to the embodiment of the present invention.

FIG. 3 is a flowchart illustrating a method for obtaining subject information according to the embodiment of the present invention.

FIG. 4 is a sequence diagram illustrating obtaining of data according to the embodiment of the present invention.

FIG. 5 is a flowchart illustrating a method for obtaining subject information according to another embodiment of the present invention.

FIG. 6 is a schematic diagram at a time when optical characteristic information is observed in NPL 1.

### DESCRIPTION OF EMBODIMENTS

In measurement using photoacoustic imaging, a probe might unintentionally move during the measurement. In this case, observation regions of optical characteristic information obtained as a result of the measurement undesirably become different before and after the movement of the probe. Therefore, when an arithmetic mean of a plurality of pieces of optical characteristic information is obtained as disclosed in NPL 1, an arithmetic mean of a plurality of pieces of optical characteristic information in different regions is undesirably obtained. This problem also arises if a subject moves during the measurement.

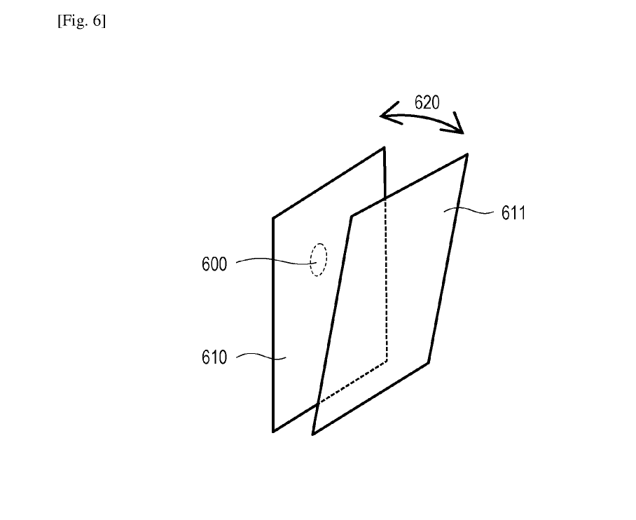

FIG. 6 is a schematic diagram at a time when optical characteristic information is observed using a subject information obtaining apparatus according to NPL 1.

For example, if a probe or a subject moves in an elevation direction 620 while optical characteristic information in an observation region 610 is being observed as illustrated in FIG. 6, a region in which the optical characteristic information is obtained changes from the observation region 610 to an observation region 611. In this case, a photoacoustic wave source 600 existing in the observation region 610 does not exist in the observation region 611. Therefore, if an arithmetic mean of the observation region 610 and the observation region 611 is obtained, pieces of optical characteristic information in different regions are combined. As a result, the quantitativity of resultant optical characteristic information undesirably decreases.

Not only when the probe or the subject moves in the elevation direction 620 but also when observation regions become different before and after movement, the quantitativity of resultant optical characteristic information decreases.

### First Embodiment

Therefore, a subject information obtaining apparatus according to this embodiment first obtains optical characteristic information from photoacoustic signal data obtained by receiving a photoacoustic wave in a first period. Furthermore, the subject information obtaining apparatus according to this embodiment obtains morphological information from echo signal data obtained by transmitting and receiving an acoustic wave in the first period. Here, the morphological information refers to information obtained from echo signal data obtained by transmitting and receiving an acoustic wave. For example, the morphological information may be a B-mode image representing the echo intensity of a transmitted acoustic wave as distribution, a Doppler image representing the velocity distribution of the internal structure of a subject, an elastographic image representing the elasticity distribution (distortion factor, shear wave velocity, and Young's modulus) of the internal structure of a subject, speckle pattern data caused by scattering in a subject, or the like.

Next, the subject information obtaining apparatus according to this embodiment obtains optical characteristic information from photoacoustic signal data obtained by receiving a photoacoustic wave in a second period. Furthermore, the subject information obtaining apparatus according to this embodiment obtains morphological information from echo signal data obtained by transmitting and receiving an acoustic wave in the second period.

Next, the subject information obtaining apparatus according to this embodiment obtains similarity between a plurality of pieces of morphological information obtained in a plurality of periods. The subject information obtaining apparatus according to this embodiment then combines a plurality of pieces of optical characteristic information obtained in the same periods as the plurality of pieces of morphological information if the similarity is equal to or higher than a certain value.

Here, “high similarity” indicates that it is likely that a plurality of pieces of morphological information are based on a plurality of pieces of echo signal data that have been obtained in the same region. The similarity between a plurality of pieces of optical characteristic information obtained in the same periods as a plurality of pieces of morphological information is the same as the similarity of the plurality of pieces of morphological information.

Because the intensity of an echo, which is a reflected wave of an acoustic wave, is typically higher than the intensity of a photoacoustic wave, the intensity of echo signal data is higher than the intensity of photoacoustic signal data.

In addition, the repetition frequency of radiation of light for generating photoacoustic waves is restricted by the maximum permissible exposure (MPE). Therefore, the repetition frequency of radiation of light is typically lower than the repetition frequency of transmission and reception of acoustic waves. Accordingly, the number of pieces of echo signal data obtained in a certain period of time is larger than the number of pieces of photoacoustic signal data obtained in the certain period of time.

For this reason, with respect to the quantitativity of information obtained from data obtained in a certain period of time, the quantitativity of morphological information is typically higher than the quantitativity of optical characteristic information. Therefore, the accuracy of the similarity between a plurality of pieces of morphological information is typically higher than the accuracy of the similarity between a plurality of pieces of optical characteristic information. That is, the reliability of the similarity between a plurality of pieces of morphological information is high.

As described above, the subject information obtaining apparatus according to this embodiment may select pieces of optical characteristic information to be combined on the basis of the similarity between a plurality of pieces of morphological information. Therefore, according to the subject information obtaining apparatus according to this embodiment, it is likely that a plurality of pieces of optical characteristic information based on a plurality of pieces of photoacoustic signal data obtained in the same region may be combined.

### Basic Configuration of Subject Information Obtaining Apparatus

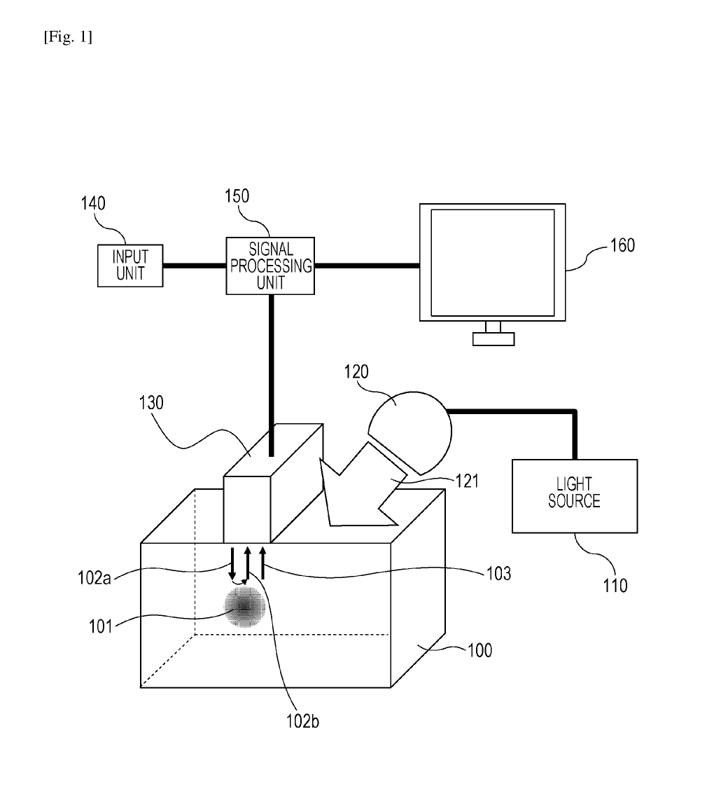

FIG. 1 is a schematic diagram illustrating the subject information obtaining apparatus according to this embodiment. The subject information obtaining apparatus illustrated in FIG. 1 includes a light source 110, an optical system 120, a transducer 130, a signal processing unit 150 as a computer, and a display unit 160.

The transducer 130 according to this embodiment has a function as a photoacoustic wave reception unit that receives photoacoustic waves generated inside a subject 100, a function as a photoacoustic wave transmission unit that transmits acoustic waves to the subject 100, and a function as an echo reception unit that receives echoes reflected inside the subject 100.

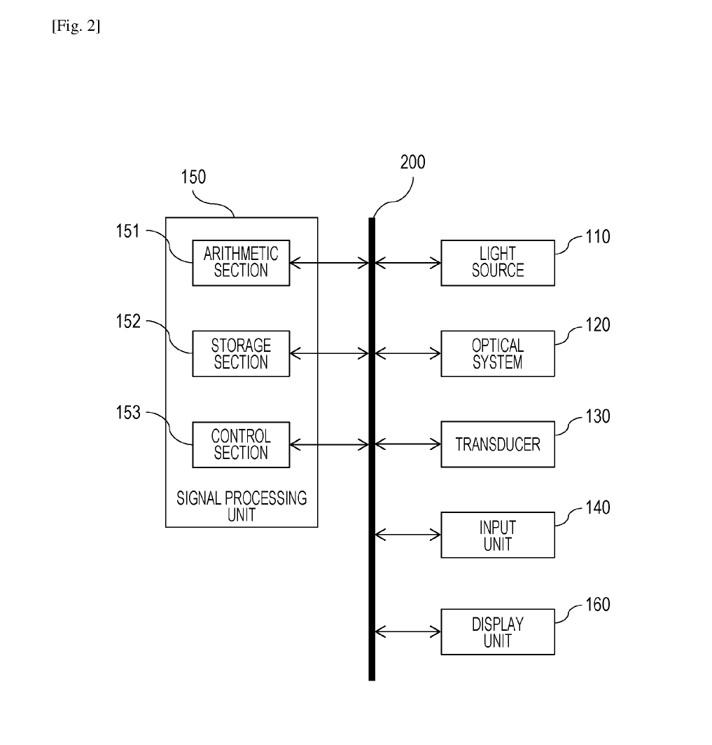

FIG. 2 is a schematic diagram illustrating details of the signal processing unit 150 and the configuration of components around the signal processing unit 150. The signal processing unit 150 includes an arithmetic section 151, a storage section 152, and a control section 153.

The control section 153 controls the operation of the components of the subject information obtaining apparatus through a bus 200. In addition, the control section 153 reads a program that is saved in the storage section 152 and in which a method for obtaining subject information, which will be described later, is described, and causes the subject information obtaining apparatus to execute the method for obtaining subject information.

The configuration of the subject information obtaining apparatus according to this embodiment will be described hereinafter.

### Subject 100 and Light Absorber 101

The subject 100 and a light absorber 101 are not components of the subject information obtaining apparatus in the present invention, but will be described hereinafter. The subject information obtaining apparatus in the present invention, for example, diagnoses malignant tumors and angiopathy of humans and animals and the like and observes the progress of chemical treatment. Therefore, examples of the subject 100 include portions of breasts, necks, abdomens of living bodies, that is, humans and animals, to be diagnosed.

The light absorber 101 in the subject 100 is a portion of the subject 100 whose light absorption coefficient is relatively high. When a human body is the subject 100, the light absorber 101 may be a malignant tumor containing oxyhemoglobin, deoxyhemoglobin, or blood vessels or newly formed blood vessels including a large amount of oxyhemoglobin or deoxyhemoglobin. Plaque on a carotid artery wall or the like may also be the light absorber 101.

### Light Source 110

As the light source 110, a pulse light source capable of generating nanosecond or microsecond pulse light can be used. More specifically, a pulse light source capable of generating light whose pulse width is about 10 nanoseconds can be used in order to efficiently generate photoacoustic waves. The wavelength of a light source to be used can be a wavelength at which light is able to reach the inside of the subject 100. More specifically, when the subject 100 is a living body, the wavelength may be 500 nm to 1,200 nm.

As a light source, a laser or a light-emitting diode may be used. For example, as a laser, one of various types of lasers such as a solid-state laser, a gas laser, a dye laser, and a semiconductor laser may be used.

### Optical System 120

Light emitted from the light source 110 may be processed by the optical system 120 in such a way as to have a desired light distribution shape and guided to the subject 100. In the optical system 120, optical components such as, for example, a mirror that reflects light, a lens that changes the shapes of beams by focusing or diffusing light, a diffusing plate that diffuses light, and an optical fiber that propagates light may be used. Any optical components may be used insofar as the light emitted from the light source 110 may be radiated onto the subject 100 as desired light.

If the light emitted from the light source 110 may be guided to the subject 100 as desired light, the optical system 120 need not be used.

### Transducer 130

The transducer 130 receives photoacoustic waves and acoustic waves such as echoes, and converts the received waves into electrical signals, which are analog signals. In addition, the transducer 130 may transmit acoustic waves. Any device may be used as the transducer 130, such as one that utilizes a piezoelectric phenomenon, one that utilizes optical resonance, or one that utilizes changes in capacitance, insofar as acoustic waves may be transmitted and received.

The transducer 130 may include a plurality of transducers arranged in an array.

The transducer 130 may simultaneously have a function as a photoacoustic wave reception unit that receives photoacoustic waves generated inside the subject 100, a function as an acoustic wave transmission unit that transmits acoustic waves to the subject 100, and a function as an ultrasonic wave reception unit that receives echoes reflected inside the subject 100. In this case, it becomes easier to receive acoustic waves in the same region and reduce the areas occupied by the components.

Alternatively, a plurality of transducers may have the above-described functions, respectively. In this case, the plurality of transducers having the above-described functions may be collectively referred to as the transducer 130 according to this embodiment.

### Input Unit 140

The input unit 140 is a member configured in such a way as to enable a user to specify desired information in order to input the desired information to the signal processing unit 150. As the input unit 140, a keyboard, a mouse, a touch panel, a dial, buttons, or the like may be used. When a touch panel is used as the input unit 140, the display unit 160 may be the touch panel that also serves as the input unit 140.

### Signal Processing Unit 150

As illustrated in FIG. 2, the signal processing unit 150 includes the arithmetic section 151, the storage section 152, and the control section 153.

The arithmetic section 151 typically includes a device such as a central processing unit (CPU), a graphics processing unit (GPU), an amplifier, an analog-to-digital (A/D) converter, a field-programmable gate array (FPGA), or an application-specific integrated circuit (ASIC). The arithmetic section 151 may include a plurality of devices instead of including a single device. Processes performed in the method for obtaining subject information according to this embodiment may be performed by any device.

The storage section 152 includes a medium such as a read-only memory (ROM), a random-access memory (RAM), or a hard disk. The storage section 152 may include a plurality of media instead of including a single medium.

The control section 153 typically includes a device such as a CPU.

The arithmetic section 151 may amplify electrical signals obtained from the transducer 130 and convert the electrical signals from analog signals into digital signals.

In addition, the arithmetic section 151 may obtain optical characteristic information regarding the subject 100 by performing a process based on an image reconfiguration algorithm on photoacoustic signal data.

As the image reconfiguration algorithm for obtaining optical characteristic information, for example, reverse projection in a time domain or a Fourier domain, which is generally used in tomography, may be used. When it is possible to take time to perform reconfiguration, an image reconfiguration method such as reverse problem solving realized by an iterative process may be used.

In the photoacoustic imaging, however, when post-reception focusing is performed using a transducer including an acoustic lens or the like, optical characteristic information regarding the subject 100 may be obtained without performing the image reconfiguration. In this case, the arithmetic section 151 need not perform the process based on the image reconfiguration algorithm.

Alternatively, the arithmetic section 151 may obtain morphological information regarding the subject 100 by performing the process based on the image reconfiguration algorithm on echo signal data. For example, as an image reconfiguration algorithm for obtaining a B-mode image, a delay addition process for matching the phases of signals or the like may be used. In addition, as an image reconfiguration algorithm for obtaining a Doppler image, a process for calculating changes in frequency between a transmitted wave and a received wave or the like may be used. In addition, as an image reconfiguration algorithm for obtaining an elastographic image, a process for calculating distortion in each sound ray of data obtained before and after deformation of tissue or the like may be used.

The arithmetic section 151 can be configured in such a way as to be able to simultaneously perform pipeline processing on a plurality of pieces of data. In this case, the time taken to obtain subject information may be reduced.

The processes performed in the method for obtaining subject information may be saved in the storage section 152 as a program to be executed by the control section 153. However, the storage section 152 in which the program is saved is a nonvolatile recording medium such as a ROM.

The signal processing unit 150 and the transducer 130 may be provided inside the same case. However, a signal processing unit stored in the same case as the transducer 130 may perform part of signal processing, and a signal processing unit provided outside the case may perform the rest of the signal processing. In this case, the signal processing units provided inside and outside the case in which the transducer 130 is stored may be collectively referred to as the signal processing unit 150 according to this embodiment.

(Display Unit 160)

The display unit 160 is a device that displays optical characteristic information or morphological information output from the signal processing unit 150. The display unit 160 is typically a liquid crystal display, but may be a display of another type, namely a plasma display, an organic electroluminescent (EL) display, or a field emission display (FED). Alternatively, the display unit 160 may be provided separately from the subject information obtaining apparatus in the present invention.

### Method for Obtaining Subject Information

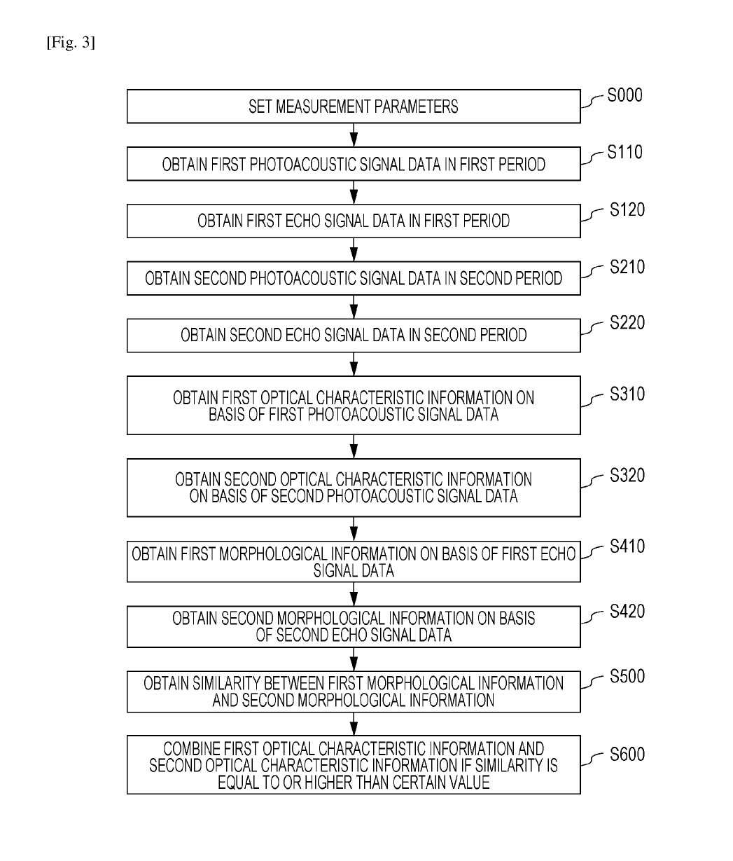

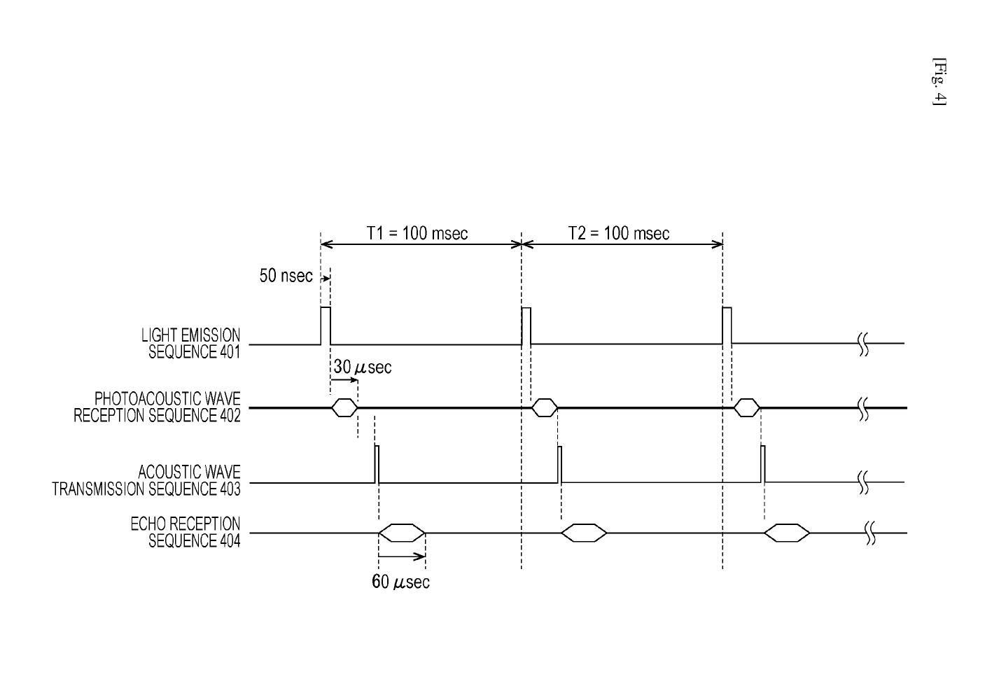

Next, the method for obtaining subject information according to this embodiment in which the subject information obtaining apparatus illustrated in FIGS. 1 and 2 is used will be described with reference to FIGS. 3 and 4. FIG. 3 is a flowchart illustrating the method for obtaining subject information according to this embodiment. FIG. 4 is a sequence diagram illustrating obtaining of photoacoustic signal data and echo signal data according to this embodiment. The method illustrated in FIG. 3 and the obtaining illustrated in FIG. 4 are executed by the control section 153.

(S000: Step of Setting Measurement Parameters)

In this step, measurement parameters are set and saved to the storage section 152. Here, the measurement parameters include parameters relating to all measurement environments for obtaining subject information.

The user may arbitrarily set the measurement parameters using the input unit 140. Alternatively, the measurement parameters may be set in advance before shipment.

For example, as the measurement parameters, conditions under which light used for measurement is radiated (wavelength, pulse width, power, and the like), the type of optical characteristic information to be obtained, the type of morphological information to be obtained, and the like may be set.

In addition, as the measurement parameters, the number of pieces of photoacoustic signal data used for obtaining one frame of optical characteristic information and the number of pieces of echo signal data used for obtaining one frame of morphological information may be set. That is, the numbers of times that measurement in S110, S120, S210, and S220 is performed are set as the measurement parameters.

In addition, as the measurement parameters, the number of frames of optical characteristic information to be obtained and the number of frames of morphological information to be obtained may be set. That is, the numbers of times that operations in S310 and S410 are performed may be set as the measurement parameters. In this embodiment, the operations in S310 and S410 are each performed twice, and two frames of optical characteristic information and two frames of morphological information are obtained.

In addition, a certain value that serves as a threshold for similarity may be set as one of the measurement parameters. The threshold can be determined on the basis of a slice width including the characteristics of the acoustic lens in an elevation direction or the like.

In addition, the number of frames of optical characteristic information to be combined may be set as one of the measurement parameters. That is, the number of frames of optical characteristic information to be used for combining may be set as one of the measurement parameters, the similarity between those frames being determined to be high in S600, which will be described later.

(S110: Step of Obtaining First Photoacoustic Signal Data in First Period)

In this step, first, light 121, which is first light, emitted from the light source 110 is radiated onto the subject 100 through the optical system 120 in a first period T1. The radiated light 121 is absorbed by the light absorber 101, which momentarily expands to generate a photoacoustic wave 103, which is a first photoacoustic wave. In this embodiment, as indicated by a light emission sequence 401 illustrated in FIG. 4, the control section 153 controls the light source 110 such that the light source 110 emits the light 121 having a pulse width of 50 ns, in order to generate the photoacoustic wave 103.

Next, the transducer 130 receives the photoacoustic wave 103 and converts the photoacoustic wave 103 into an electrical signal, which is a first photoacoustic signal, and then outputs the electrical signal to the signal processing unit 150. In this embodiment, as indicated by a photoacoustic wave reception sequence 402 illustrated in FIG. 4, the control section 153 controls the transducer 130 such that the transducer 130 receives the photoacoustic wave 130 for 30 microseconds. The reception time is determined in accordance with a depth at which optical characteristic information is to be observed.

Next, the arithmetic section 151 performs certain processing such as amplification and A/D conversion on the electrical signal output from the transducer 130, and stores the electrical signal subjected to the certain processing in the storage section 152 as first photoacoustic signal data.

Here, the photoacoustic signal data in this embodiment refers to data used for obtaining optical characteristic information, which will be described later. The photoacoustic signal data in this embodiment is a concept that includes data obtained without performing the certain processing on the electrical signal output from the transducer 130 and stored in the storage section 152.

In this embodiment, as indicated by the light emission sequence 401 illustrated in FIG. 4, the control section 153 controls the light source 110 such that the repetition frequency of radiation of light by the light source 110 becomes 10 Hz. Because each period of the repetition frequency is set as the first period T1, the first period T1 is 100 ms.

Alternatively, a plurality of pieces of photoacoustic signal data obtained by radiating light a plurality of times in the first period T1 may be collectively referred to as the first photoacoustic signal data. In this case, a plurality of pieces of photoacoustic signal data obtained in this step may be added and used as the first photoacoustic signal data. On the other hand, after the plurality of pieces of photoacoustic signal data are obtained in this step, the arithmetic section 151 may obtain a plurality of pieces of optical characteristic information from the plurality of pieces of photoacoustic signal data in S310, which will be described later. In this case, the plurality of pieces of optical characteristic information may be added and used as first optical characteristic information.

(S120: Step of Obtaining First Echo Signal Data in First Period)

In this step, the transducer 130 transmits an ultrasonic wave 102a, which is a first acoustic wave, to the subject 100 in the first period T1. When the transmitted ultrasonic wave 102a is reflected inside the subject 100, an echo 102b, which is a first echo, is generated.

Next, the transducer 130 receives the echo 102b, converts the echo 102b into an electrical signal, which is a first echo signal, and outputs the electrical signal to the signal processing unit 150. In this embodiment, as indicated by an acoustic wave transmission sequence 403 and an echo reception sequence 404 illustrated in FIG. 4, the control section 153 controls the transducer 130 such that the transducer 130 transmits the acoustic wave 102a and receives the echo 102b for 60 microseconds. The reception time is determined in accordance with a depth at which morphological information obtained from an echo is to be observed, a safety index for ultrasonic waves, and the like.

Here, as the safety index, for example, spatial-peak temporal-average intensity (ISPTA; <720 mW/cm²) in a Food and Drug Administration (FDA) standard or the like may be used. Because the ISPTA is determined by a time average of maximum values of intensity of transmitted acoustic waves, the ISPTA is proportional to time intervals of transmission. Therefore, when ultrasonic waves are transmitted and received by the same transducer as in this embodiment, the reception time can be set in consideration of sufficient pulse repetition frequency (PRF) that satisfies safety requirements.

Next, the arithmetic section 151 performs processing such as amplification and A/D conversion on the electrical signal, and stores the electrical signal subjected to the processing in the storage section 152 as first echo signal data.

The echo signal data in this embodiment refers to data used for obtaining morphological information, which will be described later. The echo signal data in this embodiment is a concept that includes data obtained without performing the processing on the electrical signal output from the transducer 130 and stored in the storage section 152.

A plurality of pieces of echo signal data obtained by transmitting and receiving an acoustic wave a plurality of times in the first period T1 may be used as the first echo signal data. In this case, the arithmetic section 151 may store the plurality of pieces of echo signal data that have been obtained in the storage section 152, or may add the plurality of pieces of echo signal data and store the plurality of pieces of echo signal data in the storage section 152.

In addition, the control section 153 may control the light source 110 and the transducer 130 such that the radiation of light in S110 and the transmission of an ultrasonic wave in S120 are simultaneously performed. In this case, since the speed of light in a subject is typically higher than the speed of an ultrasonic wave, an echo, which is a reflected wave of a transmitted ultrasonic wave, reaches the transducer 130 after a photoacoustic wave generated by radiating light reaches the transducer 130. Therefore, a photoacoustic wave and an echo generated at a particular position may be received at different time points, which makes it possible to distinguish their respective reception signals on the basis of the reception time points. Furthermore, since light and an ultrasonic wave may be simultaneously output, a photoacoustic wave and an echo may be efficiently received in a limited period of time.

In addition, when a photoacoustic wave and an echo are simultaneously received, their respective reception signals need to be separated from each other. The separation of the reception signals may be realized by a process for separating frequencies performed by hardware such as a band-pass filter or software executed by the signal processing unit 150 while utilizing a difference between the frequencies of the photoacoustic wave and the echo.

(S210: Step of Obtaining Second Photoacoustic Signal Data in Second Period)

In this step, second photoacoustic signal data is obtained by receiving a second photoacoustic wave generated when second light is radiated onto the subject 100 in a second period T2. In this step, the second photoacoustic signal data is obtained in the same manner as in S110.

When the concentration of a substance (for example, hemoglobin concentration in blood, oxygen saturation of blood, or the like) in the subject 100 is to be obtained using the first photoacoustic signal data and the second photoacoustic signal data, the first light and the second light need to be radiated using different wavelengths. In this case, the same light source 110 may be used for these wavelengths, or a plurality of light sources 110 corresponding to these wavelengths may be used.

(S220: Step of Obtaining Second Echo Signal Data in Second Period)

In this step, second echo signal data is obtained by transmitting a second acoustic wave and receiving a second echo, which is generated when the second acoustic wave is reflected inside the subject 100, in the second period T2. In this step, the second echo signal data is obtained in the same manner as in S210.

The control section 153 may control the light source 110 and the transducer 130 such that the radiation of light in S210 and the transmission of an ultrasonic wave in S220 are simultaneously performed.

(S310: Step of Obtaining First Optical Characteristic Information on Basis of First Photoacoustic Signal Data)

In this step, the arithmetic section 151 obtains first initial sound pressure distribution, which is the first optical characteristic information regarding the inside of the subject 100, by performing image reconfiguration on the first photoacoustic signal data.

When the arithmetic section 151 is to obtain light absorption coefficient distribution as the first optical characteristic information in this step, the light amount distribution of the first light in the subject 100 needs to be obtained in addition to the first initial sound pressure distribution obtained by performing the image reconfiguration. In this case, for example, the arithmetic section 151 may calculate the light amount distribution by analyzing a light propagation model described in NPL 2, or may read a light amount distribution table stored in the storage section 152 in advance. At this time, the arithmetic section 151 may refer to the conditions under which light is radiated stored in the storage section 152 as measurement parameters.

In addition, when a plurality of pieces of photoacoustic signal data have been obtained in S110, the arithmetic section 151 may obtain optical characteristic information from each of the plurality of pieces of photoacoustic signal data. The arithmetic section 151 may then add the plurality of pieces of photoacoustic characteristic information and use the resultant photoacoustic characteristic information as the first optical characteristic information.

(S320: Step of Obtaining Second Optical Characteristic Information on Basis of Second Photoacoustic Signal Data)

In this step, the arithmetic section 151 obtains second initial sound pressure distribution, which is second optical characteristic information regarding the inside of the subject 100, by performing image reconfiguration on the second photoacoustic signal data stored in the storage section 152.

In S320, the second photoacoustic characteristic information may be obtained in the same manner as in S310.

(S410: Step of Obtaining First Morphological Information on Basis of First Echo Signal Data)

In this step, the arithmetic section 151 obtains a first B-mode image, which is first morphological information regarding the inside of the subject 100, by performing image reconfiguration on the first echo signal data stored in the storage section 152.

When a plurality of pieces of echo signal data have been obtained in S120, the arithmetic section 151 may obtain morphological information on the basis of each of the plurality of pieces of echo signal data. The arithmetic section 151 may then compound the plurality of pieces of morphological information and use the resultant morphological information as the first morphological information.

(S420: Step of Obtaining Second Morphological Information on Basis of Second Echo Signal Data)

In this step, the arithmetic section 151 obtains a second B-mode image, which is second morphological information regarding the inside of the subject 100, by performing image reconfiguration on the second echo signal data stored in the storage section 152.

In S420, the second morphological information may be obtained in the same manner as in S410.

(S500: Step of Obtaining Similarity between First Morphological Information and Second Morphological Information)

In this step, the arithmetic section 151 obtains the similarity between the first morphological information obtained in S410 and the second morphological information obtained in S420 using one of methods that will be described later. The obtained similarity is stored in the storage section 152. In this embodiment, the similarity is calculated using the first morphological information as a reference frame.

The similarity is typically calculated by obtaining a correlation coefficient.

Here, as a method for obtaining a correlation coefficient, one of various known methods such as the sum of absolute differences (SAD), the sum of squared differences (SSD), cross-correlation (CC), normalized cross-correlation (NCC), and zero-mean normalized cross-correlation (ZNCC) may be used.

For example, if the SAD is used, the arithmetic section 151 may calculate correlation coefficients S_{SAD} using the following expression, in which pixels in blocks of two images are denoted by f(i, j) and g(i, j), respectively.

$$\begin{matrix}[ Math . 1 ] & \\ {S}_{SAD} = {\sum }_{i} {\sum }_{j} \vert f ( i , j ) - g ( i , j ) \vert & ( Expression 1 )\end{matrix}$$

Alternatively, the arithmetic section 151 may calculate the correlation coefficients S SAD by applying a full-search algorithm to a plurality of pieces of second morphological information g(i+x, j+y), where x is equal to or larger than −5 but smaller than or equal to 5 and y is equal to or larger than −5 but smaller than or equal to 5, or the like whose positions have been moved relative to the first morphological information f(i, j). Alternatively, the arithmetic section 151 may obtain the correlation coefficients S_{SAD} by applying a known search algorithm to the second morphological information using part of the first morphological information as a reference in order to reduce calculation time.

The closer the correlation coefficients S_{SAD} are to zero, the higher is the similarity between the first morphological information and the second morphological information. Therefore, a value closest to zero is used as an index in a corresponding block region. When the SAD is used, the values of similarity may be reciprocals of the correlation coefficients S_{SAD}.

Next, for example, if the SSD is used, the arithmetic section 151 may calculate correlation coefficients S_{SSD} using the following expression, in which the pixels in the blocks of the two images are denoted by f(i, j) and g(i, j), respectively. In this case, the closer the correlation coefficients S_{SSD} are to zero, the higher is the similarity between the first morphological information and the second morphological information. When the SSD is used, the values of similarity may be reciprocals of the correlation coefficients S_{SSD}.

$$\begin{matrix}[ Math . 2 ] & \\ {S}_{SSD} = {\sum }_{i} {\sum }_{j} {\{ f ( i , j ) - g ( i , j ) \}}^{2} & ( Expression 2 )\end{matrix}$$

Next, for example, if the CC is used, the arithmetic section 151 may calculate correlation coefficients S_{CC} using the following expression, in which the pixels in the blocks of the two images are denoted by f(i, j) and g(i, j), respectively. In this case, the larger the correlation coefficients S_{CC}, the higher the similarity between the first morphological information and the second morphological information. When the CC is used, the values of similarity may be the correlation coefficients S_{CC}.

$$\begin{matrix}[ Math . 3 ] & \\ {S}_{CC} = {\sum }_{i} {\sum }_{j} f ( i , j ) - g ( i , j ) & ( Expression 3 )\end{matrix}$$

Next, for example, if the NCC is used, the arithmetic section 151 may calculate correlation coefficients S_{NCC} using the following expression, in which the pixels in the blocks of the two images are denoted by f(i, j) and g(i, j), respectively. In this case, the closer the correlation coefficients S_{NCC} are to 1, the higher is the similarity between the first morphological information and the second morphological information. When the NCC is used, the values of similarity may be the correlation coefficients S_{NCC}.

$$\begin{matrix}[ Math . 4 ] & \\ {S}_{NCC} = \frac{{\sum }_{i} {\sum }_{j} f ( i , j ) - g ( i , j )}{\sqrt{{\sum }_{i} {\sum }_{j} {f ( i , j )}^{2} \times {\sum }_{i} {\sum }_{j} {g ( i , j )}^{2}}} & ( Expression 4 )\end{matrix}$$

Next, for example, if the ZNCC is used, the arithmetic section 151 may calculate correlation coefficients S_{ZNCC} using the following expression, in which the pixels in the blocks of the two images are denoted by f(i, j) and g(i, j), respectively. Here, f (with a line above) in Expression 5 denotes an average in the region f(i, j), and g (with a line above) denotes an average in the region g(i, j). In this case, the closer the correlation coefficients S_{ZNCC} are to 1, the higher is the similarity between the first morphological information and the second morphological information. When the ZNCC is used, the values of similarity may be the correlation coefficients S_{ZNCC}.

$$\begin{matrix}[ Math . 5 ] & \\ {S}_{ZNCC} = \frac{{\sum }_{i} {\sum }_{j} ( ( f ( i , j ) - \overline{f} ) ( g ( i , j ) - \overline{g} ) )}{\sqrt{{\sum }_{i} {\sum }_{j} {f ( i , j - \overline{f} )}^{2} \times {\sum }_{i} {\sum }_{j} {g ( i , j - \overline{g} )}^{2}}} & ( Expression 5 )\end{matrix}$$

Alternatively, the arithmetic section 151 may calculate the correlation coefficients by applying a full-search algorithm to the second morphological information using the first morphological information as a reference. Alternatively, the arithmetic section 151 may obtain the correlation coefficients by applying a known search algorithm to the second morphological information using part of the first morphological information as a reference in order to reduce the calculation time.

Alternatively, in order to make the calculation faster, the calculation may be performed using a Fourier transform without directly calculating the correlation coefficients. For example, first, the arithmetic section 151 Fourier transforms signals of the two images, and obtains a complex conjugate for one of the signals subjected to the Fourier transform. The arithmetic section 151 may then obtain the correlation coefficients by multiplying the signals subjected to the Fourier transform and inverse Fourier transforming a generated cross-spectrum.

Alternatively, statistical test values may be used as the correlation coefficients. For example, a P value obtained by performing a chi-square test on image data groups whose positions are different from each other may be used as the similarity between the blocks of the two images.

Alternatively, the arithmetic section 151 may obtain the correlation coefficients by performing interpolation or correction between correlation coefficients of regions located close to one another. Furthermore, the arithmetic section 151 may obtain the correlation coefficients of regions that are smaller than a certain pixel (voxel in the case of three dimensions) by performing interpolation or correction between the correlation coefficients of regions located closed to one another.

(S600: Step of Combining First Optical Characteristic Information and Second Optical Characteristic Information When Similarity is Equal to or Higher Than Threshold)

In this step, first, the arithmetic section 151 determines whether or not the similarity obtained in S500 is equal to or higher than the threshold set in S000. If the similarity is equal to or higher than the threshold, the arithmetic section 151 combines the first optical characteristic information obtained in S310 and the second optical characteristic information obtained in S320. The resultant optical characteristic information is saved to the storage section 152.

Next, the arithmetic section 151 performs a process for obtaining image data such as luminance conversion on the resultant optical characteristic information to convert the optical characteristic information into image data. The arithmetic section 151 then outputs the image data to the display unit 160 to cause the display unit 160 to display the optical characteristic information as an image. In the method for obtaining subject information according to this embodiment, however, the step of displaying the optical characteristic information on the display unit 160 is not mandatory.

“Combining optical characteristic information” in this embodiment refers to obtaining a single piece of new optical characteristic information from a plurality of pieces of optical characteristic information.

For example, a plurality of pieces of optical characteristic information may be combined using an arithmetic mean method, a geometric mean method, or a harmonic mean method on the plurality of pieces of optical characteristic information.

When the wavelengths of the first light and the second light are different, the arithmetic section 151 may obtain the concentration of a substance in the subject 100 by combining the first optical characteristic information and the second optical characteristic information. That is, “combining optical characteristic information” in this embodiment also refers to obtaining the concentration of a substance in the subject 100 from a plurality of pieces of optical characteristic information.

Alternatively, the arithmetic section 151 may obtain the resultant optical characteristic information by multiplying a plurality of frames of optical characteristic information by corresponding weighting values and combining the plurality of frames of optical characteristic information.

If the similarity is lower than the threshold, the first optical characteristic information and the second optical characteristic information are not used for the combining. In this case, the optical characteristic information that are saved in the storage section 152 but has not been used for the combining may be deleted. Alternatively, the optical characteristic information that has not been used for the combining may be overwritten when new optical characteristic information is saved to the storage section 152. Thus, by deleting unnecessary optical characteristic information from the storage section 152, the amount of memory used in the storage section 152 may be reduced.

In addition, the arithmetic section 151 may display the number of frames of optical characteristic information used for the combining on the display unit 160. When the number of frames of optical characteristic information to be used for the combining has been set in S000, the arithmetic section 151 may cause the display unit 160 to display a difference between the number of frames set and the number of frames actually used for the combining or a ratio of the number of frames set to the number of frames actually used for the combining.

In addition, in one frame of optical characteristic information, the arithmetic section 151 need not use regions whose similarities are lower than the threshold as targets of the combining and may use only regions whose similarities are equal to or higher than the threshold as targets of the combining Therefore, frames used for the combining may differ between regions of optical characteristic information obtained as a result of the combining In this case, the display unit 160 may display frames used for the combining in each region, the number of frames used, and the like.

In addition, the display unit 160 may be configured in such a way as to be able to display pieces of optical characteristic information at a time before the combining and optical characteristic information obtained as a result of the combining by switching display between these pieces of optical characteristic information.

As described above, by using the method for obtaining subject information according to this embodiment, pieces of optical characteristic information based on pieces of photoacoustic signal data that are likely to have been obtained in the same region may be selectively combined, which makes it likely to increase the quantitativity of resultant optical characteristic information.

In this embodiment, S120 is performed after S110, and S220 is performed after S210. Therefore, the first period in this embodiment refers to a period from a time at which the first light is radiated to generate the first photoacoustic wave to a time at which the second light is radiated to generate the second photoacoustic wave.

In addition, the first period in this embodiment refers to a period in which measurement for obtaining the first optical characteristic information and the first morphological information is performed. That is, the first period refers to a period obtained by combining a period in which the first light is radiated and the first photoacoustic wave is received and a period in which the first acoustic wave is transmitted and the first echo is received.

The second period in this embodiment refers to a period in which measurement for obtaining the second optical characteristic information and the second morphological information is performed. That is, the second period refers to a period obtained by combining a period in which the second light is radiated and the second photoacoustic wave is received and a period in which the second acoustic wave is transmitted and the second echo is received.

In this embodiment, S110 may be performed after S120, and S210 may be performed after S220. In this case, the first period refers to a period from a time at which the first acoustic wave is transmitted to generate the first echo to a time at which the second acoustic wave is transmitted to generate the second echo.

In addition, in this embodiment, the method for obtaining subject information may be executed not in the two periods, namely the first period and the second period, but in three or more periods. That is, three or more frames of morphological information and three or more frames of optical characteristic information may be obtained. The three or more frames of morphological information and three or more frames of optical characteristic information may be used in S500 and S600, respectively.

In addition, if the number of frames reaches the number of frames to be obtained set in S000, the arithmetic section 151 need not save information obtained thereafter to the storage section 152. In doing so, unnecessary information is not saved to the storage section 152, thereby reducing the amount of memory used in the storage section 152.

In addition, in this embodiment, the first period and the second period may overlap.

In addition, morphological information for obtaining the similarity and optical characteristic information to be combined may be used in the method for obtaining subject information according to this embodiment insofar as the morphological information and the optical characteristic information are correlated with each other. That is, even if morphological information and optical characteristic information are obtained in different periods, the optical characteristic information may correspond to the morphological information or the morphological information may correspond to the optical characteristic information.

### Second Embodiment

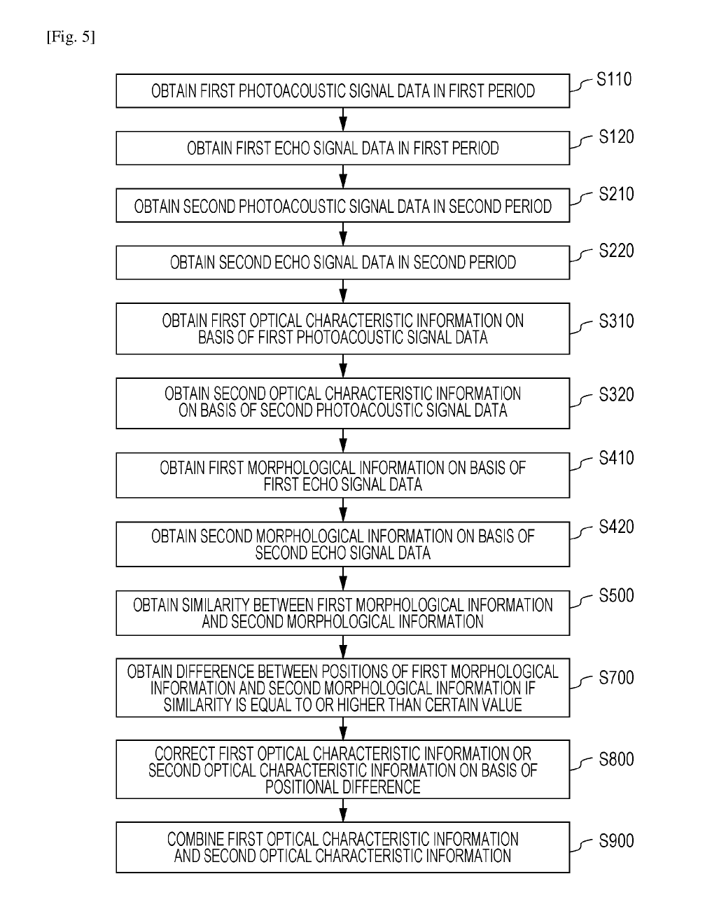

Next, a method for obtaining subject information according to a second embodiment will be described with reference to a flowchart of FIG. 5. Among steps illustrated in FIG. 5, the same steps as those illustrated in FIG. 2 are given the same reference numerals, and description thereof is omitted. In this embodiment, too, the subject information obtaining apparatus used in the first embodiment, which is illustrated in FIGS. 1 and 2, is used. The flowchart of FIG. 5 is executed by the control section 153. In this embodiment, the control section 153 executes steps S000 to S500 as in the first embodiment.

(S700: Step of Obtaining Difference between Positions of First Morphological Information and Second Morphological Information When Similarity is Equal to or Higher Than Threshold)

In this step, the arithmetic section 151 determines whether or not the similarity obtained in S500 is equal to or higher than the threshold. Next, if the similarity is equal to or higher than the threshold, the arithmetic section 151 obtains a difference between the position of the first morphological information obtained in S410 and the position of the second morphological information obtained in S420. The obtained difference is saved to the storage section 152.

Here, as an algorithm for calculating the difference, a known method such as a block-matching algorithm or an affine transformation algorithm may be applied to a plurality of pieces of morphological information. For example, the block-matching algorithm is an algorithm that divides a certain frame of morphological information that serves as a reference into small regions (blocks) having a certain size, that detects which part of other frames each block corresponds to, and that calculates differences between the positions of the corresponding blocks as movement vectors.

For example, when the block-matching algorithm is applied, a movement vector between each block of the reference frame and a block with which the similarity is highest may be obtained as a difference between the positions of corresponding blocks. If the calculated difference is different from positional differences of nearby blocks, a value estimated by performing interpolation or the like on the basis of the positional differences of the nearby blocks may be used, instead.

After obtaining positional differences of all frames of the morphological information, the arithmetic section 151 may determine pieces of optical characteristic information to be combined by comparing the similarity with the threshold. However, as in this embodiment, the arithmetic section 151 can determine whether or not to obtain positional differences on the basis of the similarity after obtaining the correlation coefficients in S500. In this case, a step of obtaining positional differences of pieces of morphological information corresponding to pieces of optical characteristic information that are not the targets of the combining may be reduced. That is, the time taken to complete the method for obtaining subject information may be reduced.

When positional differences are obtained on the basis of the similarity, the arithmetic section 151 may obtain positional differences using the similarity obtained in S500. In doing so, a step of newly obtaining the similarity separately from S500 in order to obtain positional differences on the basis of the similarity may be omitted.

(S800: Step of Correcting Coordinates of First Optical Characteristic Information or Coordinates of Second Optical Characteristic Information on Basis of Positional Difference)

In this step, the arithmetic section 151 moves the coordinates of the first optical characteristic information obtained in S310 or the coordinates of the second optical characteristic information obtained in S320 by the positional difference obtained in S700. At this time, a direction in which the coordinates are moved is determined on the basis of the direction of the positional difference obtained in S700.

For example, when the difference between the position of a frame of the first optical characteristic information, which serves as the reference, and the position of a frame of the second optical characteristic information has been obtained from the movement vector using the block-matching algorithm, the arithmetic section 151 may move the coordinates of the first optical characteristic information by the movement vector.

(S900: Step of Combining First Optical Characteristic Information and Second Optical Characteristic Information)

In this step, the arithmetic section 151 combines the first optical characteristic information and the second optical characteristic information subjected to the correction in S800. In this step, as in S600, the arithmetic section 151 may combine a plurality of pieces of optical characteristic information using a method such as the arithmetic mean method, the geometric mean method, or the harmonic mean method.

As described above, according to the method for obtaining subject information according to this embodiment, pieces of optical characteristic information based on pieces of photoacoustic signal data that are likely to have been obtained from the same region may be selectively combined, which makes it likely to increase the quantitativity of resultant optical characteristic information.

Furthermore, according to the method for obtaining subject information according to this embodiment, pieces of optical characteristic information may be combined after a difference between the positions of the pieces of optical characteristic information are corrected, which increases the quantitativity of resultant optical characteristic information.

In both of the above embodiments, an example has been described in which pieces of optical characteristic information are combined if the similarity between a plurality of corresponding morphological information is equal to or higher than the threshold. In the present invention, however, a plurality of pieces of optical characteristic information may be combined if the similarity between the plurality of pieces of optical characteristic information is equal to or higher than the threshold. In this case, it becomes more likely to be able to combine the plurality of pieces of optical characteristic information based on pieces of photoacoustic signal data in the same region.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiments of the present invention, and by a method performed by the computer of the system or apparatus by, for example, reading out and executing the computer executable instructions from the storage medium to perform the functions of one or more of the above-described embodiments. The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-286686, filed Dec. 28, 2012, which is hereby incorporated by reference herein in its entirety.

1. A subject information obtaining apparatus comprising:
a light source configured to emit light;
a photoacoustic wave reception unit configured to receive a photoacoustic wave generated when the light is radiated onto a subject and output a photoacoustic signal;
an acoustic wave transmission unit configured to transmit an acoustic wave to the subject;
an echo reception unit configured to receive an echo of the acoustic wave and output an echo signal; and
a signal processing unit configured to obtain a plurality of pieces of optical characteristic information regarding the subject on the basis of the photoacoustic signal and a plurality of pieces of morphological information regarding the subject on the basis of the echo signal,
wherein the signal processing unit obtains similarity between the plurality of pieces of morphological information, and
wherein, if the similarity is equal to or higher than a certain value, the signal processing unit combines the plurality of pieces of optical characteristic information corresponding to the plurality of pieces of morphological information.

2. The subject information obtaining apparatus according to claim 1, wherein, if the similarity is lower than the certain value, the signal processing unit does not combine the plurality of pieces of optical characteristic information corresponding to the plurality of pieces of morphological information.

3. The subject information obtaining apparatus according to claim 1, wherein the signal processing unit obtains correlation coefficients of the plurality of pieces of morphological information, and obtains the similarity on the basis of the correlation coefficients.

4. The subject information obtaining apparatus according to claim 3, wherein the signal processing unit obtains the correlation coefficients using one of the following methods: a sum of absolute differences, a sum of squared differences, cross-correlation, normalized cross-correlation, and zero-mean normalized cross-correlation.

5. The subject information obtaining apparatus according to claim 1,
wherein, if the similarity is equal to or higher than the certain value, the signal processing unit obtains differences between positions of the plurality of pieces of morphological information, and
wherein the signal processing unit corrects coordinates of the plurality of pieces of optical characteristic information corresponding to the plurality of pieces of morphological information on the basis of the differences between the positions of the plurality of pieces of morphological information.

6. The subject information obtaining apparatus according to claim 5, wherein the signal processing unit obtains correlation coefficients of the plurality of pieces of morphological information and obtains the differences on the basis of the correlation coefficients.

7. The subject information obtaining apparatus according to claim 1, wherein the signal processing unit combines the plurality of pieces of optical characteristic information by performing one of the following methods: an arithmetic mean method, a geometric mean method, and a harmonic mean method.

8. The subject information obtaining apparatus according to claim 1, wherein the light source emits light beams having a plurality of wavelengths, wherein the signal processing unit obtains the plurality of pieces of optical characteristic information on the basis of the light beams having the plurality of wavelengths, and wherein the signal processing unit obtains concentration of a substance in the subject by combining the plurality of pieces of optical characteristic information.

9. The subject information obtaining apparatus according to claim 1, wherein the acoustic wave transmission unit and the echo reception unit include the same transducer.

10. The subject information obtaining apparatus according to claim 1, wherein the photoacoustic wave reception unit, the acoustic wave transmission unit, and the echo reception unit include the same transducer.

11. A method for controlling a subject information obtaining apparatus, the method comprising the steps of:
obtaining a photoacoustic signal by receiving a photoacoustic wave generated when light is radiated onto a subject;
transmitting an acoustic wave to the subject;
obtaining an echo signal by receiving an echo of the acoustic wave;
obtaining a plurality of pieces of optical characteristic information regarding the subject on the basis of the photoacoustic signal;
obtaining a plurality of pieces of morphological information regarding the subject on the basis of the echo signal,
obtaining similarity between the plurality of pieces of morphological information; and
combining, if the similarity is equal to or higher than a certain value, the plurality of pieces of optical characteristic information corresponding to the plurality of pieces of morphological information.

12. A non-transitory computer-readable storage medium which records a program for causing a computer to execute the method for controlling a subject information obtaining apparatus according to claim 11.

13. A subject information obtaining apparatus comprising:
a light source configured to emit light;
a photoacoustic wave reception unit configured to receive a photoacoustic wave generated when the light is radiated onto a subject and output a photoacoustic signal; and
a signal processing unit configured to obtain a plurality of pieces of optical characteristic information regarding the subject on the basis of the photoacoustic signal,
wherein the signal processing unit obtains similarity between the plurality of pieces of optical characteristic information, and
wherein, if the similarity is equal to or higher than a certain value, the signal processing unit combines the plurality of pieces of optical characteristic information.

14. A subject information obtaining apparatus comprising:
a light source configured to emit light;
a photoacoustic wave reception unit configured to receive a photoacoustic wave generated when the light is radiated onto a subject and output a photoacoustic signal;
an acoustic wave transmission unit configured to transmit an acoustic wave to the subject;
an echo reception unit configured to receive an echo of the acoustic wave and output an echo signal; and
a signal processing unit configured to obtain a plurality of pieces of optical characteristic information regarding the subject on the basis of the photoacoustic signal and a plurality of pieces of morphological information regarding the subject on the basis of the echo signal,
wherein the signal processing unit obtains first optical characteristic information regarding the subject on the basis of a first photoacoustic signal output from the photoacoustic wave reception unit that has received a photoacoustic wave in a first period,
wherein the signal processing unit obtains first morphological information regarding the subject on the basis of a first echo signal output from the echo reception unit that has received an echo of an acoustic wave in the first period, wherein the signal processing unit obtains second optical characteristic information regarding the subject on the basis of a second photoacoustic signal output from the photoacoustic wave reception unit that has received a photoacoustic wave in a second period, which is different from the first period, wherein the signal processing unit obtains second morphological information regarding the subject on the basis of a second echo signal output from the echo reception unit that has received an echo of an acoustic wave in the second period,
wherein the signal processing unit obtains similarity between the first morphological information and the second morphological information, and wherein, if the similarity is equal to or higher than a certain value, the signal processing unit combines the first optical characteristic information and the second optical characteristic information.

15. The subject information obtaining apparatus according to claim 14, wherein the light source radiates first light in the first period and second light in the second period, and wherein the acoustic wave transmission unit transmits a first acoustic wave in the first period and a second acoustic wave in the second period.
